(19) United States
(12) Patent Application Publication
(10) Pub. No.: US 2018/0036148 A1
(43) Pub. Date: Feb. 8, 2018

(54) ENERGY-HARVESTING MESOFLUIDIC IMPULSE PROSTHESIS

(71) Applicant: Orthocare Innovations LLC, Edmonds, WA (US)

(72) Inventors: Lucas Samuel Lincoln, Seattle, WA (US); David Alan Boone, Seattle, WA (US); Nicholas Roy Corson, Mukilteo, WA (US)

(73) Assignee: Orthocare Innovations LLC, Edmonds, WA (US)

(21) Appl. No.: 15/550,652

(22) PCT Filed: Feb. 13, 2015
(86) PCT No.: PCT/US15/15884

§ 371 Date: Aug. 11, 2017

(51) Int. Cl. A61F 2/68; A61B 5/00; A61B 5/103

(52) U.S. Cl. CPC A61F 2/68; A61B 5/1036; A61B 5/4851; A61F 2/64

(57) ABSTRACT

A prosthetic joint including a hydraulic system, comprising: at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the hydraulic system; a load-determining sensor; a displacement-determining sensor; and a microprocessor configured to actuate one or more flow controllers based upon a load determining sensor input, a displacement-determining sensor input, a product of the load-determining sensor input and the displacement-determining sensor input, any time derivative thereof, or any combination thereof, wherein one or more flow controllers are configured to control displacing fluid from the chamber to the accumulator during periods of a threshold negative work, and one or more flow controllers are configured to control displacing fluid from the accumulator to the chamber to perform positive work.

### STATEMENT OF U.S. GOVERNMENT LICENSE RIGHTS

This invention was made with U.S. Government support under R43HD080309 awarded by National Institutes of Health. The U.S. Government has certain rights in the invention.

### BACKGROUND

Prosthesis users have adapted to the limitations of current prostheses by abnormal changes to gait which have been shown to result in reduced metabolic efficiency and increased loading at more proximal joints and on the sound limb. Long-term deleterious effects include skin breakdown on the residual limb, and overloading of the intact limb (gait asymmetry) with subsequent osteoarthritis.

A normal-functioning human ankle controls energy throughout the gait cycle, acting in turn as a dissipater, storage device, power producer, or energy neutral component. The ankle can produce as much as five times more work than is dissipated, but this is not required for many activities of daily living. Creating precisely timed impulses at the ankle joint is important to natural gait and motion, and active movement is particularly important for perceived effort, comfort, and stumble prevention. It would be advantageous for prostheses to similarly provide power output at specific moments in the gait cycle.

However, most current prosthetic ankles are either rigid bodies, transferring joint function to motion proximally and distally, or they generally function as springs. This latter type of flexible prosthetic feet have carbon fiber keels that initially bend in a plantarflexion direction, returning some energy as the tibial segment accelerates forward in early stance, then bend toward dorsiflexion, absorbing power during midstance, and then recoil toward plantarflexion again in late stance and pre-swing. Since these springs are passive, they do not return energy past the neutral position of the spring.

### SUMMARY

Methods of harvesting and selectively reapplying energy to a prosthetic joint are disclosed. The methods may include providing a prosthetic joint. The prosthetic joint may include at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the chamber, accumulator and reservoir; a load-determining sensor; a displacement-determining sensor; a microprocessor configured to actuate one or more flow controllers based upon a load-determining sensor input, a displacement-determining sensor input, a product of the load-determining sensor input and the displacement-determining sensor input, any time derivative thereof, or any combination thereof. The method further includes displacing fluid from the chamber to the accumulator during periods of a threshold negative work on the joint; and displacing fluid from the accumulator to the chamber to allow the joint to perform positive work.

In some embodiments, the method may further include displacing fluid from the chamber to the reservoir during periods below the threshold negative work.

In some embodiments, the prosthetic joint is an ankle joint, and the ankle joint is connected to a prosthetic foot and a pylon, wherein the ankle joint allows rotation of the prosthetic foot with respect to the pylon.

In some embodiments, the method may further include determining a flow controller state by determining a swing positioning state, a controlled plantarflexion state, a controlled dorsiflexion state, and a powered plantarflexion state.

In some embodiments, the method may further include storing energy in the accumulator during controlled dorsiflexion, or during controlled plantarflexion, or during both, and returning energy during powered plantarflexion.

In some embodiments, the method may further include returning energy during swing positioning.

In some embodiments, swing positioning includes dorsiflexing the foot and elevating the toe.

In some embodiments, the method may further include determining conditions to transition from the swing positioning state to the controlled plantarflexion state, conditions to transition from the controlled plantarflexion state to the controlled dorsiflexion state, conditions to transition from the controlled dorsiflexion state to the powered plantarflexion state, and conditions to transition from the powered plantarflexion state to the swing positioning state.

In some embodiments, in the swing positioning state, fluid is displaced from a posterior accumulator to a posterior chamber, and fluid is displaced from an anterior chamber to an anterior reservoir; in the controlled plantarflexion state, fluid is displaced from the posterior chamber to the posterior accumulator, and fluid is displaced from the anterior reservoir to the anterior chamber; in the controlled dorsiflexion state, fluid is displaced from the posterior reservoir to the posterior chamber, and fluid is displaced from the anterior chamber to the anterior accumulator; and in the powered plantarflexion state, fluid is displaced from the posterior chamber to the posterior reservoir, and fluid is displaced from the anterior accumulator to the anterior chamber.

In some embodiments, the threshold negative work is performed when a limb connected to the joint is applied on a ground surface to generate a ground reaction force greater than a pressure in the accumulator.

In some embodiments, the flow controllers include one or more automatically operated shut-off valves.

In some embodiments, the method further includes passing fluid through a restrictor when displacing fluid from the chamber to the reservoir.

In some embodiments, the method further includes producing the negative work above the threshold by contacting a limb connected to the joint on a surface to generate a ground reaction force.

In some embodiments, the displacement-determining sensor is an angle-determining sensor.

In some embodiments, the joint is a prosthetic knee joint.

In some embodiments, the method further includes storing energy in the accumulator when sitting from a standing position and returning energy when standing from a sitting position.

In some embodiments, the method further includes storing energy in the accumulator during descending and returning energy during ascending.

In some embodiments, the flow controllers are pulsed open during displacing fluid from the accumulator to the chamber.

Methods of harvesting energy from a first joint and selectively reapplying the energy to a second joint are disclosed. The methods include providing an energy-harvesting hydraulic system including at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the system; a load-determining sensor; a displacement-determining sensor; a microprocessor configured to actuate one or more flow controllers based upon a load-determining sensor input, a displacement-determining sensor input, a product of the load-determining sensor input and the displacement-determining sensor input, any time derivative thereof, or any combination thereof. The methods include displacing fluid from the chamber to the accumulator during periods of a threshold negative work on a first joint; and displacing fluid from the accumulator to the chamber to allow a second joint to perform positive work.

In some embodiments, the first joint is an ankle and the second joint is a knee, or the first joint is the knee and the second joint is the ankle.

Prosthetic joints are disclosed. The joints may include a hydraulic system, including at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the hydraulic system; a load-determining sensor; a displacement-determining sensor; and a microprocessor configured to actuate one or more flow controllers based upon a load-determining sensor input, a displacement-determining sensor input, a product of the load-determining sensor input and the displacement-determining sensor input, any time derivative thereof, or any combination thereof, wherein one or more flow controllers are configured to control displacing fluid from the chamber to the accumulator during periods of a threshold negative work, and one or more flow controllers are configured to control displacing fluid from the accumulator to the chamber to perform positive work.

In some embodiments, the joint may further include a piston in the chamber, wherein a limb is actuated by the piston during displacing fluid from the accumulator to the chamber.

In some embodiments, the limb actuates the piston during displacing fluid from the chamber to the accumulator.

In some embodiments, the joint may further include a cam and cam follower, wherein the cam follower is in contact with the cam, and the cam follower is connected to the piston.

In some embodiments, the cam includes an involute cam surface.

In some embodiments, the joint further includes a pivot, wherein the pivot rotates a first prosthetic limb with respect to a second prosthetic limb.

In some embodiments, the first prosthetic limb is a prosthetic foot, and the second prosthetic limb includes a pylon and socket.

In some embodiments, the joint further includes a first and second accumulator, a first and second reservoir, and a first and second chamber, wherein the first and second chambers are placed on opposite sides of a pivot, and the first chamber includes flow paths to the first accumulator and the first reservoir, and the second chamber includes flow paths to the second accumulator and the second reservoir.

In some embodiments, a fluid flow path from each chamber to the accumulator includes, in parallel, an automatically operated shut-off valve and a check valve, wherein the check valve is configured to allow flow from the chamber to the accumulator and obstruct flow from the accumulator to the chamber.

In some embodiments, a fluid flow path from each chamber to the reservoir includes an automatically operated shut-off valve and, in parallel, a restrictor and a check valve, wherein the check valve is configured to allow flow from the reservoir to the chamber and obstruct flow from the chamber to the reservoir.

In some embodiments, the load-determining sensor is a strain gauge.

In some embodiments, the load-determining sensor is a pressure transducer.

In some embodiments, the displacement-determining sensor is a potentiometer.

In some embodiments, the displacement-determining sensor is a hall effect sensor.

In some embodiments, the flow controllers include a solenoid valve.

Prosthetic joints are disclosed that may include a first and second connector and a pivot device that allows the first and second connector to rotate with respect to each other, wherein the first connector is configured to attach to a first prosthetic member and the second connector is configured to attach to a second prosthetic member; a first and second chamber, wherein the chambers are disposed on opposite sides of the pivot device; a first and second piston positioned in the first and second chamber, wherein the pistons are positioned to actuate the rotation of the joint; an accumulator configured to store hydraulic fluid at a high pressure, wherein the accumulator connects to each chamber through a flow path including, in parallel, a shut-off valve and a check valve, wherein the check valve is configured to allow flow from each respective chamber to the accumulator and obstruct flow from the accumulator to each respective chamber; a reservoir configured to store hydraulic fluid at a low pressure, wherein the reservoir connects to each chamber through a flow path including a shut-off valve; a load-determining sensor; a displacement-determining sensor; a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from one chamber at a time to the accumulator during periods of a threshold negative work on the joint, and displacing fluid from the accumulator to one chamber at a time to allow the joint to perform positive work.

Prosthetic joints are disclosed that may include a first and second connector and a pivot device that allows the first and second connector to rotate with respect to each other, wherein the first connector is configured to attach to a first prosthetic member and the second connector is configured to attach to a second prosthetic member; a first and second chamber, wherein the chambers are disposed on opposite sides of the pivot device; a first and second piston positioned in the first and second chamber, wherein the pistons are positioned to actuate the rotation of the joint; a first and second accumulator configured to store hydraulic fluid at a high pressure, wherein the first accumulator connects to the first chamber through a flow path including, in parallel, a shut-off valve and a check valve, wherein the check valve is configured to allow flow from the first chamber to the first accumulator and obstruct flow from the first accumulator to the first chamber, and the second accumulator connects to the second chamber through a flow path including, in parallel, a shut-off valve and a check valve, wherein the check valve is configured to allow flow from the second chamber to the second accumulator and obstruct flow from the second accumulator to the second chamber; a first and second reservoir configured to store hydraulic fluid at a low pressure, wherein the first reservoir connects to the first chamber through a flow path including, in parallel, shut-off valve and a check valve configured to allow flow from the first reservoir to the first chamber and obstruct flow from the first chamber to the first reservoir, and the second reservoir connects to the second chamber through a flow path including a shut-off valve and a check valve configured to allow flow from the second reservoir to the second chamber and obstruct flow from the second chamber to the second reservoir; a load-determining sensor; a displacement-determining sensor; a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from each chamber to the respective accumulator during periods of a threshold negative work on the joint, and for displacing fluid from each accumulator to the respective chamber to allow the joint to perform positive work.

Prosthetic joints are disclosed that may include a hydraulic system including: at least one chamber; at least one accumulator configured to store hydraulic fluid at a high pressure; at least one reservoir configured to store hydraulic fluid at a low pressure; one or more fluid flow paths connecting the chamber to the accumulator and reservoir, and flow controllers in the fluid flow paths; and hydraulic fluid in the system. The joints may further include a load-determining sensor; a displacement-determining sensor; a microprocessor to actuate the flow controllers based upon a load-determining sensor input, a displacement-determining sensor, any product of the load-determining sensor input and the displacement-determining sensor input, any time derivative thereof, or any combination thereof, wherein the flow controllers are configured to displace fluid from the chamber to the accumulator during periods of a threshold negative work, and the flow controllers are configured to displace fluid from the accumulator to the chamber to perform positive work, and wherein the threshold negative work is performed on a first joint and the positive work is performed by a second joint different from the first joint.

Some embodiments of the prosthetic joints include flow controllers that are further configured to displace fluid from the chamber to the reservoir during periods below the threshold negative work.

The mechanical and hydraulic design of the energy-harvesting ankle is such that inherent mechanical properties are responsible for the bulk of the control intelligence, minimizing sensor requirements, electronic complexity, and cost. Furthermore, the inherent passive stability of the ankle joint and control system limits its potential to injure the user, providing clear benefits with respect to ensuring the safety of the amputee users for which it is intended.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of one embodiment of an energy-harvesting system for a prosthesis;

FIG. 2 is a diagrammatical illustration of one embodiment of an energy-harvesting system for a prosthesis;

FIG. 3 is a diagrammatical illustration of one embodiment of an energy-harvesting system for a prosthesis;

FIG. 4 is a diagrammatical illustration of one embodiment of an energy-harvesting system incorporated into a prosthetic joint and limbs;

FIG. 5 is a diagrammatical illustration of an energy-harvesting prosthesis in various phases of gait;

FIG. 6 is a finite state diagram of an energy-harvesting system; and

FIG. 7 is a diagrammatical illustration of an energy-harvesting system distributed in two prosthetic joints and limbs.

### DETAILED DESCRIPTION

When people lose a limb due to illness or injury, a prosthesis may bring back some functionality and mobility to the person. With the loss of a limb comes the loss of muscle to move the limb. Accordingly, prosthesis can greatly benefit from having powered limbs.

A joint is any rotating element that can connect to two members or limbs. For example, an ankle joint connects the lower leg to the foot, a knee joint connects the lower leg to the upper leg, an elbow connects the lower arm to the upper arm, a hip joint connects the upper leg to the pelvis, and so on.

A prosthetic joint can move by application of an external force acting on one of the two limbs connected to the joint. Many prosthetic joints are only passive. That is, the joint is moved only when acted upon by external forces, such as when applying weight on the joint. Powered joints rely on batteries to power actuators that in turn power the limbs. In contrast to the joints that derive power from batteries, the joints disclosed herein derive power to move limbs from energy stored in a hydraulic system. The hydraulic systems described herein can allow joints to store energy during periods of negative work, and then, release the energy at selected periods when desired to power a limb. The hydraulic system also allows the joint to operate as a dampened joint during periods when the joint is neither storing energy nor being powered.

Work is the product of force and displacement. Thus, when a joint and limb are moved via the application of external forces, the joint experiences negative work, i.e., work done on the joint. The hydraulic systems described herein can use a fluid-filled system including a chamber with piston and a high pressure accumulator to store some of the energy during periods of negative work. The hydraulic systems described herein can then release the energy to the chamber from the high pressure accumulator, thus, causing the joint to do positive work and move a limb. During periods other than storing energy or releasing energy from the high pressure accumulator, fluid can be exchanged between the chamber and a reservoir. Periods during which negative work is produced so that energy may be harvested include periods during walking, particularly during periods in the stance phase. Other times for harvesting energy may include sitting from a standing position or vice versa.

A description of an ankle joint is used to describe several aspects of this disclosure. However, it is not meant to be limiting. It is intended that the energy-harvesting hydraulic systems can be used in other joints, such as the knee, the hip, the shoulder, the elbow, the wrist, or a finger joint. In some cases, the energy-harvesting system may use the work harvesting using one joint, and then, release the energy to another joint. For example, the ankle joint can be used to harvest energy during walking, but then, the energy is released at a different joint, such as the knee, or vice versa.

The behavior of an ankle during the stance phase of level walking is characterized by two major periods: power absorption from heel contact through full weight acceptance and power release as the ankle plantarflexes during the transition to toe-off. During swing, the ankle dorsiflexes so that the toe does not impact the ground as the leg swings to full extension in preparation for the next heel contact. The energy-harvesting system can store energy within one or more high pressure accumulators during the power absorption phase of support and then return a portion of this stored energy to plantarflex the ankle prior to toe-off while reserving the remainder for dorsiflexion of the ankle during swing. Alternatively, the energy can be used for powering a different joint. Control is achieved by first decomposing ankle torque during support and swing into passive impedances. Functional motion is then attained by switching the limb between the support and swing impedances as the subject progresses through the locomotive function.

Stance phase may be decomposed into three sub-phases: Controlled plantarflexion (CPF), controlled dorsiflexion (CDF), and powered plantarflexion (PPF). The healthy human ankle functions in a particular way during each of these phases and the energy-harvesting control system is intended to be biomimetic, directly emulating healthy ankle function at each stage. The swing phase (SW) is generally defined from toe-off to heel-strike (heel contact).

Use of an energy-harvesting system with an ankle joint can better replicate biofidelic loading and range of motion that may provide significant improvements in stability and locomotion efficiency. Initiation of the swing phase of gait is normally a propulsive moment in the gait cycle. With non-actuated prostheses, the user must overcome the dead-weight of the prosthesis by accelerating its mass through their prosthetic suspension. Pistoning is the displacement of the socket relative to the amputee's residual limb. Maximum pistoning is caused by initiation of swing phase, occurring at about 75% of the gait cycle, immediately following toe off. Many lower limb amputees report dissatisfaction with socket comfort, residual limb pain, and/or skin breakdown from exactly this kind of pistoning. Conversely, decreasing the displacement of the prosthesis to the amputated limb creates a more natural gait and the amputee is more likely to feel like the prosthesis is a part of their body.

The disclosed energy-harvesting systems provide harvesting of the energy normally dissipated in human locomotion, and subsequently can release the energy at an optimal timing.

With loss of a biological limb, lower limb amputees lack key features of efficient gait; such as push-off by their limb in late stance phase, dorsiflexion during early swing, and a nearly energy neutral profile over the gait cycle. The forces produced by the plantarflexors create joint moments that cause the ankle joint to rotate and produce net ankle power generation in late stance phase. Ankle power peaks in a powered-plantarflexion (PPF) event. The magnitude and timing of this powered plantarflexion impulse is part of efficient bipedal gait and is used for accelerating both the center of mass and the trailing limb into swing phase.

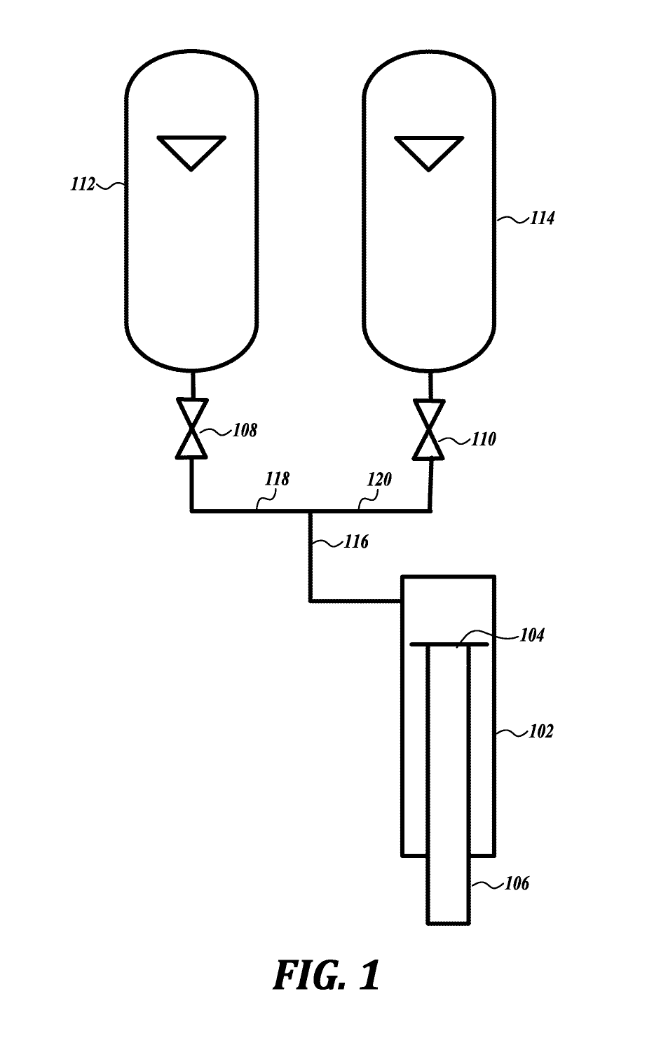

Referring to FIG. 1, a diagrammatical illustration of one embodiment of an energy-harvesting system is illustrated. The system includes a chamber 102. A piston 104 resides in and is allowed to reciprocate within the chamber. The piston 104 can be connected to a rod 106. The piston 104 and rod 106 can function as an actuator when coupled to a prosthetic limb. The space above the piston 104 is connected via line 116 to a first low pressure accumulator 112 (the reservoir) and a second high pressure accumulator 114. Low pressure accumulators, such as 112, can be referred to herein as reservoirs.

The line 116 branches into branch line 118 that connects to the reservoir 112 and the line 116 branches into branch line 120 that connects to the high pressure accumulator 114. A first valve 108 is placed in branch line 116, and a second valve 110 is placed in branch line 110. The space above the piston 104 is thus connected to the reservoir 112 via line 116 and line 118. The high pressure accumulator 114 is connected to the space above the piston 104 via line 116 and line 120. The valves 108 and 110 can be remotely electrically opened and closed, or any amount in between, via the use of a microprocessor based on inputs from sensing instruments described herein.

The high pressure accumulators herein are any vessel for storing the hydraulic fluid under pressure. Hydraulic accumulators are known. The accumulator can include a floating piston that creates a variable volume within the accumulator. Such volume can be under pressure provided by a spring or compressed gas acting on the piston. In some embodiments, the reservoir 112 is also an accumulator. However, the reservoir 112 operates at a lower pressure than the high pressure accumulator 114. The exact pressure of the accumulators and reservoirs can be adjusted based on the particular application. For example, the accumulator and reservoir pressures can be adjusted based on the weight of a person using the prosthetic joint, or based on the type of joint, for example.

The chamber 102, low pressure reservoir 102, high pressure accumulator 104, and all lines connected thereto form a closed hydraulic system. That is, no hydraulic fluid enters or leaves the system under normal operation. The piston 104 and rod 106 can function as an actuator to do work. That is, the piston 104 is connected to a limb or other moving member. The piston 104 can also be moved by external forces acting on the limb. Thus, performing negative work on the system. When the piston 104 is compressed in the chamber 102, the hydraulic fluid can be directed to either the low pressure reservoir 112 or the high pressure accumulator 114 depending on the amount of force. Sensors that can measure force, including pressure, torque, or displacement can be used to determine whether to open or close valves 108 and 110. In some embodiments, other flow controllers, such as check valves and flow restrictors can be used.

As used in this application, work has the standard definition in physics meaning the product of a load (force) applied over a displacement. Displacement can be measured in angular displacement or linear displacement. Linear displacement can be converted to angular displacement, and vice versa, by applying a formula based on the geometry of the limb configuration. “Negative work” means net work done on the energy-harvesting system and “positive work” means net work done by the energy-harvesting system. Negative work means that the system gains energy. For example, negative work is performed when a gas (or spring) is compressed in the high pressure accumulator, thus, the energy-harvesting system gains energy. When the gas (or spring) is decompressed, the energy-harvesting systems losses energy by performing positive work. For a two chamber, two accumulator system, the contributions from both the high pressure accumulator and the reservoir would need to be considered. For example, net negative work is performed when a gas (or spring) is compressed in the high pressure accumulator minus the energy that that is used by decompressing the low pressure gas (or spring) in the reservoir. Overall, the net work done is negative, meaning the system gains energy. When the high pressure accumulator is decompressed (gas or spring) and the reservoir is compressed (gas or spring), overall, the net work is positive, meaning that the system loses energy. The energy-harvesting systems use flow controllers, such as shut-off valves, flow restrictors, check valves, for example, to modulate the hydraulic fluid into and out of the chambers, accumulators, and reservoirs. The systems further include sensors that can be used to calculate periods during which it is predicted there will be negative work and periods when to perform positive work.

Referring to FIG. 1, fluid is displaced from the chamber 102 to the accumulator 114 during periods of a threshold negative work, and fluid is displaced from the accumulator 114 to the chamber 102 to allow the performance of positive work. When the negative work is below a threshold, then, the fluid is displaced from the chamber 102 to the reservoir 112. Practically, when the piston 104 is subjected to high external loads (the load exceeds the pressure of the high pressure accumulator), the valve 110 is open and the valve 108 is closed. This valve configuration allows fluid to enter the high pressure accumulator 114 through compression of the piston 104, and energy is harvested and stored in the high pressure accumulator 114. Valve 110 may be closed and valve 108 open when the piston is allowed to reciprocate and exchange fluid back and forth with the reservoir 112 under reduced or no external load. In such case, the energy-harvesting system is in passive impedance control. When the piston 104 is desired to perform work on an external limb or member, the valve 108 is closed and the valve 110 is open. This valve configuration allows the fluid in the high pressure accumulator 114 to do work on the piston 104 by expanding the piston 104 (when the external load is less than the pressure of the high pressure accumulator). Load (force) can be measured by strain gages or pressure in the chamber or elsewhere. FIG. 1 shows an energy-harvesting system including a single chamber/piston unit, a high pressure accumulator, and a low pressure reservoir. Other energy harvesting systems may include multiples of the components of FIG. 1. For example, an energy harvesting system may include two chamber/piston units, each unit communicating with a high pressure accumulator and reservoir. Such two chamber energy-harvesting systems may be used in applications of one limb pivoting with respect to a second limb, where one chamber is placed on one side of the pivot and the second chamber is placed on the opposite side of the pivot. Alternatively, energy harvesting systems may include one or more chamber/piston units, one or more high pressure accumulators, one or more low pressure reservoirs, or any combinations thereof. The energy-harvesting systems disclosed herein are not limited to a particular number of chamber/piston units in the system, nor the number of reservoirs and high pressure accumulators. Furthermore, the energy-harvesting systems are not constrained to releasing the energy to the joint from which the energy is harvested. In some cases, the energy may be harvested using one limb or joint, and the energy is released to a second limb or joint that is different from the first.

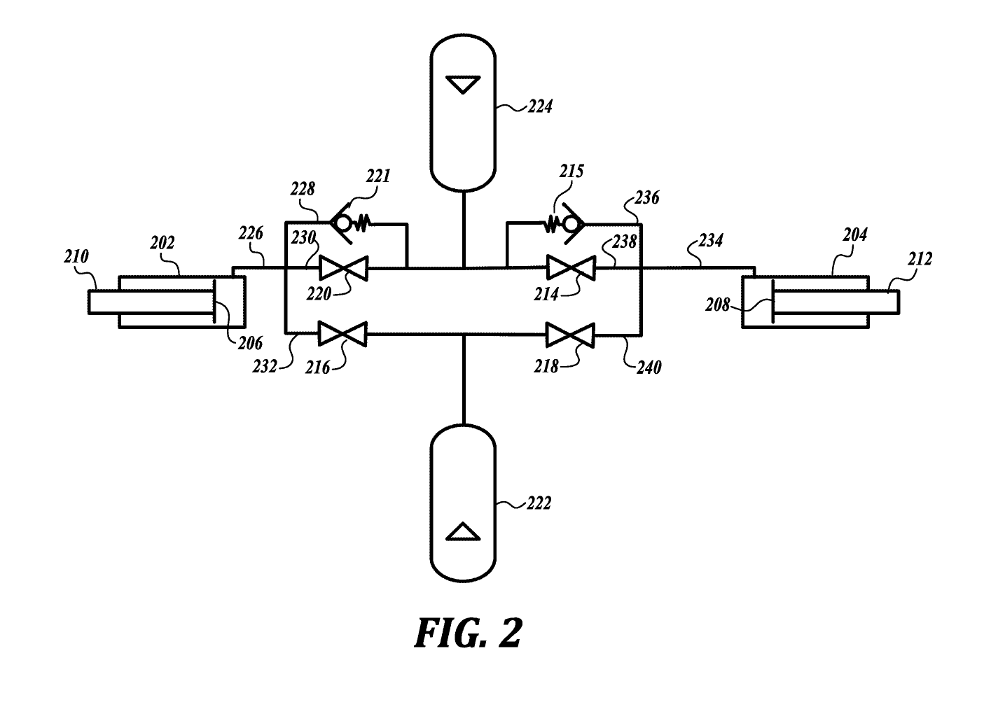

Referring to FIG. 2, another embodiment of an energy-harvesting system is illustrated. In the system of FIG. 2, a first chamber 202 and a second chamber 204 are used. First chamber 202 includes a first piston 206 connected to a first piston rod 210. Second chamber 204 includes a second piston 208 connected to a second piston rod 212. When two chamber/piston units are used, the chamber/piston units may be placed to work in opposition to each other, such as on opposite sides of the pivot or on two different joints.

Each chamber 202, 204 can connect to the same accumulator 224 and the same reservoir 222. The chambers connect to the accumulator with a flow path including, in parallel, an automatically operated shut-off valve and a check valve, wherein the check valve is configured to allow flow from the chamber to the accumulator and obstruct flow from the accumulator to the chamber. The chambers connect to the reservoir with a flow path including an automatically operated shut-off valve. The space above piston 206 connects to line 226 which branches into line 228, line 230 and line 232. Line 228 includes spring-loaded check valve 221. Line 230 includes valve 220. Line 232 includes valve 216. Lines 228 and 230 reconnect and then enter the high pressure accumulator 224. Spring-loaded check valve 221 permits flow into high pressure accumulator 224, but obstructs flow therefrom. Line 232 enters low pressure reservoir 222. The space above piston 208 connects to line 234 which branches into line 236, line 238 and line 240. Line 236 includes spring-loaded check valve 215. Line 238 includes valve 214. Line 240 includes valve 218. Lines 236 and 238 reconnect and then enter the high pressure accumulator 224. Spring-loaded check valve 215 permits flow into high pressure accumulator 224, but obstructs flow therefrom. Line 240 enters low pressure reservoir 222. High pressure accumulator 224 and low pressure reservoir 222 can be gas-charged at a high and low pressure respectively. The pistons 206 and 208 can be connected to limbs or other members to actuate the limbs or members. Depending on the placement of the chambers, the energy-harvesting system of FIG. 2 can be configured to displace fluid from the one or both chambers at the same time or sequentially to the accumulator during periods of a threshold negative work, to displace fluid from the accumulator to one or both chambers at the same time or sequentially to allow the performance of positive work, to displace fluid from one or both chambers at the same time or sequentially to the reservoir during periods below the threshold negative work, or to displace fluid from the reservoir to one or both chambers at the same time or sequentially. In some cases, when the accumulator is displacing fluid to one chamber, the other chamber is displacing fluid to the reservoir, or when one chamber is displacing fluid to the accumulator, the reservoir is displacing fluid to the other chamber. This situation can arise when one chamber is placed in opposition to the second chamber, such as one on each side of a pivot. A microcontroller can be used to open and close the appropriate valves based on input from one or more sensors described herein.

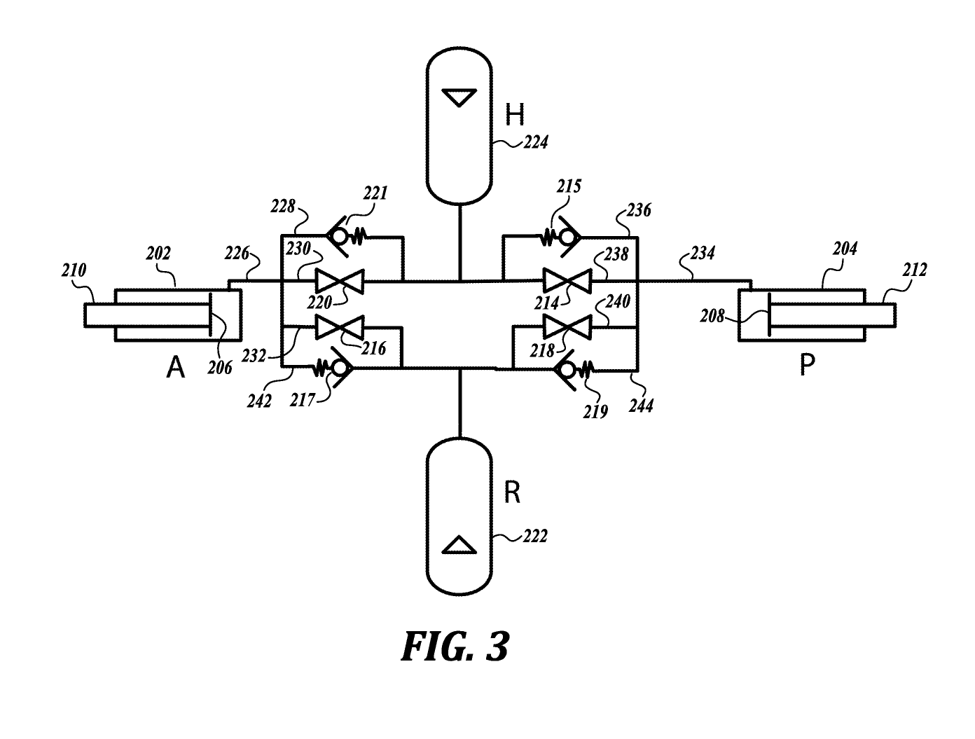

Referring to FIG. 3, another embodiment of an energy-harvesting system is illustrated. The energy-harvesting system of FIG. 3 is similar to the system of FIG. 2 except for the addition of spring-loaded check valve 217 on line 242 that permits flow out of the low pressure reservoir 222 and spring-loaded check valve 219 on line 244 that permits flow out of the low pressure reservoir. In FIG. 3, each flow path from each respective chamber to the reservoir includes, in parallel, an automatically operated shut-off valve and a check valve, wherein the check valve is configured to allow flow from the reservoir to the chamber and obstruct flow from the chamber to the reservoir. Depending on the placement of the chambers, the energy-harvesting system of FIG. 3 can be configured to displace fluid from the one or both chambers at the same time or sequentially to the accumulator during periods of a threshold negative work, to displace fluid from the accumulator to one or both chambers at the same time or sequentially to allow the performance of positive work, to displace fluid from one or both chambers at the same time or sequentially to the reservoir during periods below the threshold negative work, or to displace fluid from the reservoir to one or both chambers at the same time or sequentially. In some cases, when the accumulator is displacing fluid to one chamber, the other chamber is displacing fluid to the reservoir, or when one chamber is displacing fluid to the accumulator, the reservoir is displacing fluid to the other chamber. This situation can arise when one chamber is placed in opposition to the second chamber, such as one on each side of a pivot. A microcontroller can be used to open and close the appropriate valves based on input from one or more sensors described herein.

For an energy-harvesting system having two chamber/piston units, such as shown in FIGS. 2 and 3, the operation may generally include the following. When a piston is in compression by a load above a threshold (greater than the high pressure accumulator pressure), the hydraulic fluid is directed to the high pressure accumulator to store energy. One or both chamber/piston units can be capable of displacing hydraulic fluid to the high pressure accumulator for energy storage. Under some conditions, when a piston is in compression by a load below a threshold (less than the high pressure accumulator pressure), the hydraulic fluid is displaced to the reservoir (or a low pressure reservoir), which provides impedance control of movement of the limb (or other member). Under some conditions, when a piston is in expansion, the piston is being powered by the hydraulic fluid from the high pressure accumulator. Under some conditions, when a piston is in expansion, the piston is being moved by an external force as a consequence of the antagonistic piston being under load.

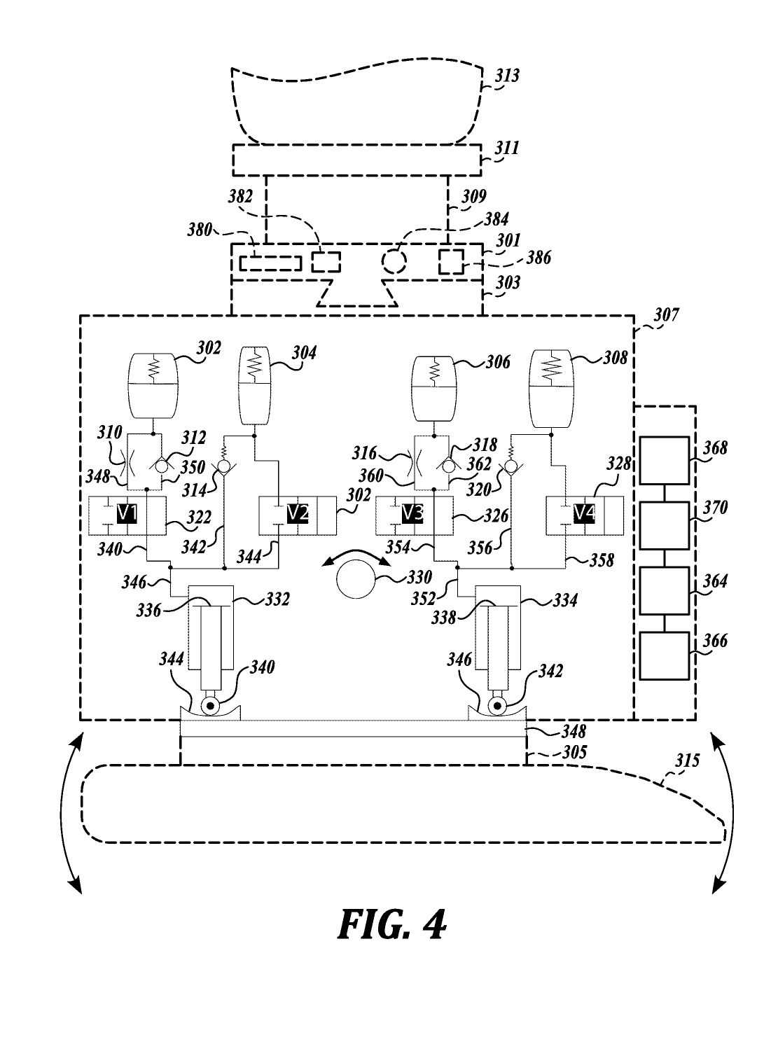

An energy-harvesting system incorporated into a prosthetic joint is diagrammatically illustrated in FIG. 4. It is to be appreciated that the energy-harvesting system of FIG. 4 is the incorporation of two energy-harvesting systems, each one resembling the energy-harvesting system of FIG. 1. That is, two chamber/piston units are placed antagonistic to each other or in direct opposition with respect to a pivoting member. It is to be appreciated that FIG. 4 is highly schematic such that the main components of the energy-harvesting system are illustrated. For purposes of illustration, the joint may be referred to as an ankle joint, however, it is not intended to limiting, as the energy-harvesting systems herein described can be incorporated into other joints.

The energy-harvesting system can be enclosed in a case defined by a broken line 307. The case 307 can include a first connector 303 and a second connector 305 configured so that the first connector 303 pivots with respect to the second connector 305. The first connector 303 can be connected to a limb or member and the second connector is connected to a second limb or member, such that there is movement of one limb with respect to the other. The joint can store energy under certain conditions and release the energy to move the limb under certain conditions as described herein. In some embodiments, the first connector 303 is further attached to a sensing device 301. The sensing device 301 can use strain gauges 380, accelerometers 382, a magnetic hall-effect encoder 384, potentiometers 386, or any combination, to sense loads, axial force, joint angle, joint angle rate of change, joint torque, joint torque rate of change being experienced by the joint. Strain gauges 380, potentiometers 386 can be used to measure load, for example. Accelerometers 382 and magnetic hall-effect encoders 384 can be used to measure displacement, including angular displacement (e.g. tilt angle, shank angle, etc.). The sensing device 301 can include a load-determining sensor or sensors and the displacement- or angle-determining sensor. A suitable sensing device can be the device known by the designation of EUROPA by Orthocare Innovations, of Mountlake Terrace in the state of Washington, USA. However, a sensing device can be assembled based on the description herein. The sensing device 301 in turn is connected to a pylon 309, and the pylon 309 is connected to a prosthetic socket 313 for receiving a lower limb. While the sensing device 301 is shown directly attached to the case 307 of the energy-harvesting system, the sensing device can be placed at the base of a prosthesis socket 313. The second connector 305 may be connected to one of a plurality of commercially available prosthetic feet 315. As can be appreciated, the joint can pivot to rotate the prosthetic foot 315 with respect to the pylon 309.

When the energy-harvesting system is used with an ankle joint, the energy harvesting system can incorporate antagonistic chambers/pistons, such as anterior and posterior with respect to the pivot 330 and foot 307. The energy-harvesting system includes a first posterior chamber 332 and a second anterior chamber 334. The posterior chamber 332 can be placed diametrically opposite the anterior chamber 334 with respect to a pivot 330. Antagonistic means that the pistons move in direct opposition. That is, when one piston is in the compression stroke, the other is in the expansion stroke. The length of stroke need not be the same for both pistons, because the distance from the pivot 330 to each piston may be different for each piston. However, in some embodiments the distance from the pivot 330 to each piston is the same.

The posterior chamber 332 includes a piston 336 connected to a piston rod which in turn is connected to a cam follower 340. The cam follower 340 is in contact with the cam surface 344. The anterior chamber 334 includes piston and piston rod 338 which in turn is connected to the cam follower 342 which makes contact with the cam surface 346. The cam surfaces or cams 344 and 346 are rigidly connected to a platform 348. The platform 348 can pivot about the pivot 330, such that plantarflexion will compress the posterior piston 336 and dorsiflexion will compress the anterior piston 338. While a cam and cam follower are shown to convert the linear motion of the actuator pistons 336, 338 into rotational motion of the joint, other mechanisms can be used, including ratchets, rack and pinion gears, and cranks. When a cam is used, the cam can be an involute cam. Two involute cams placed on opposite sides of a pivot can offer advantages.

The posterior 332 and anterior 334 chamber include flow paths with flow controllers from the respective chamber to a respective one of a high pressure accumulator and a low pressure reservoir. Chambers 332 and 334 connect to the respective accumulator with a flow path including, in parallel, an automatically operated shut-off valve and a check valve, wherein the check valve is configured to allow flow from the chamber to the accumulator and obstruct flow from the accumulator to the chamber. Chambers 332 and 334 connect to the reservoir with a flow path including an automatically operated shut-off valve, followed by, in parallel, a flow restrictor and a check valve, wherein the check valve is configured to obstruct flow from the chamber to the reservoir and allow flow from the reservoir to the chamber. Flow restrictors can include orifices, for example.

Referring to the posterior chamber 332, the space above the piston 336 connects to a line 346. The line 346 has three branch lines 340, 342, and 344. Line 340 includes shut-off valve 322. Line 344 includes shut-off valve 302. Line 340 further branches into lines 348 and 350 which then reconnect and connect to the posterior reservoir 302. Line 348 includes a restrictor 310 and line 350 includes a check valve 312. The check valve 312 only allows fluid to flow out of the low pressure reservoir 302. Line 342 includes a spring-loaded check valve 314. The spring-loaded check valve 314 only allows fluid flowing into the high pressure accumulator 304. Lines 342 and 344 connect before entering the high pressure accumulator 304. Shut-off valves 322 and 344 can be electrically driven solenoid valves.

Referring to the anterior chamber 334, the space above piston 338 connects to a line 352. Line 352 branches into three separate lines 354, 356, and 358. Line 354 includes shut-off valve 326. Line 358 includes shut-off valve 328. Line 354 further branches into lines 360 and 362. Line 360 includes a restrictor 316. Line 362 includes a check valve 318. Line 360 and 362 connect before anterior low pressure reservoir 306. Check valve 318 only allows flow out of the low pressure reservoir 306. Line 356 includes a spring-loaded check valve 320. The spring-loaded check valve 320 only allows flow into the high pressure accumulator 308. Lines 356 and 358 connect before the high pressure accumulator 308. In this disclosure, “anterior” and “posterior” may be used to associate a reservoir and accumulator with the respective anterior chamber and posterior chamber as the case may be, and should not be interpreted to mean that any reservoir or accumulator is in an anterior or posterior position. Reservoirs and accumulators may be placed in any suitable location regardless whether they are fluidly connected to an anterior or posterior chamber. Shut-off valves can be electrically driven solenoid valves.

The joint may further include a battery 368, a microprocessor 370, a memory 364, and an input/output device 366. The battery 368 can power the microprocessor 370 and the shut-off valves 322, 302, 326, and 328. The memory 364 can store instructions that command the opening and closing of the shut-off valves based on inputs from the sensing device 301. The input/output device 366 can be used to download instructions or retrieve data. The microprocessor 370 can be a Texas Instruments MSP430 microprocessor, for example. The battery 368 can be a 1200 mAh lithium polymer battery, for example, and can provide a day or more of operations per two-hour charge cycle.

Flow controllers include the shut-off valves 322, 302, 326, 328, check valves 312, 314, 318, 320, and flow restrictors 310 and 316. The microprocessor 370 is configured to actuate one or more flow controllers, primarily the shut-off valves, based upon a load-determining sensor input, a displacement-determining sensor input, a product of the load-determining sensor input and the displacement-determining sensor input, any time derivative thereof, or any combination thereof. The flow controllers are configured to displace fluid from each chamber to the respective accumulator during periods of a threshold negative work on the joint. The flow controllers are configured to displace fluid from each accumulator to the respective chamber to allow the joint to perform positive work. The flow controllers are configured to displace fluid from each chamber to the respective reservoir during periods below the threshold negative work. Because of the antagonistic nature of the chambers/pistons, when one piston is in expansion, the other is in compression. Because of the antagonistic nature of the chamber/pistons, the pistons store energy and release energy sequentially (under the right conditions). In some circumstances, the flow controllers allow for passive fluid exchange back and forth between each chamber/piston unit and the respective reservoir simultaneously for impedance control. For an energy-harvesting system having two chamber/piston units, wherein each unit has both a respective high pressure accumulator and low pressure reservoir, the operation may generally include the following. When either piston is in compression by a load, the hydraulic fluid is displaced to the respective high pressure accumulator to store energy. When a piston is in compression, the hydraulic fluid is displaced to the reservoir through a restrictor, which provides an impedance to movement of the limb (or other member). When a piston is in expansion, the piston can be powered by the hydraulic fluid from the respective high pressure accumulator. When a piston is in expansion, the piston can be moved by an external force as a consequence of the antagonistic piston being under load. The different states of operation are described herein using an ankle joint as a representative example.

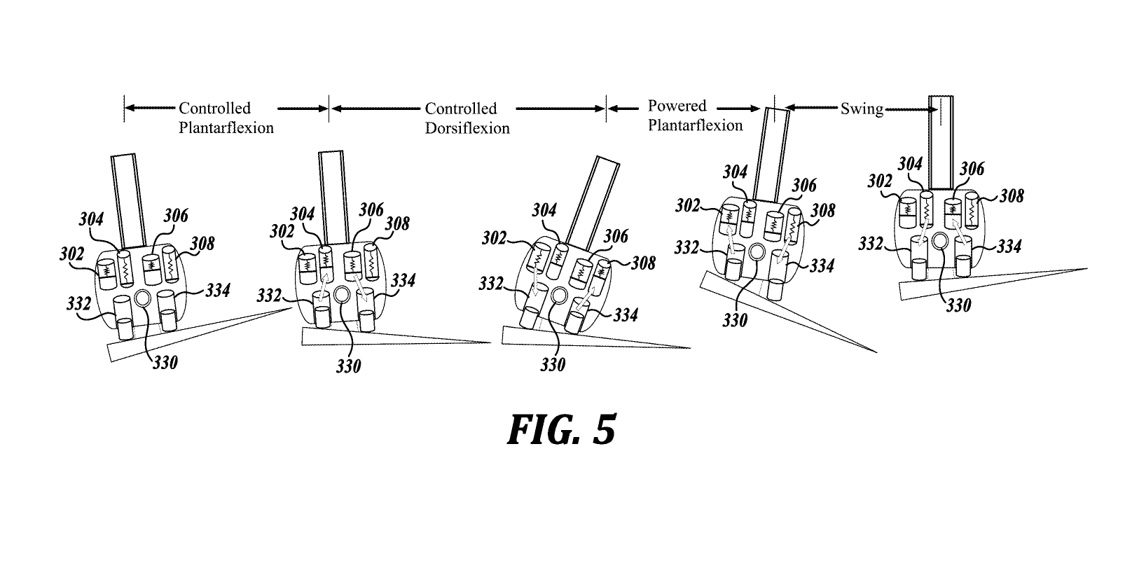

Referring to FIG. 5, the different phases of gait for one leg is depicted with the corresponding actions of the hydraulic fluid of an ankle joint with an energy-harvesting system of FIG. 4.

With an energy-harvesting ankle joint of FIG. 4, the joint can go through the different phases and dorsiflex and plantarflex a prosthetic foot to mimic the natural muscular actions of a healthy foot. The stance phase begins with heel strike. Then, a phase of controlled plantarflexion follows. Controlled plantarflexion transitions to a phase of controlled dorsiflexion before, after, or about midstance. The transition is referred to as “rollover.” After controlled dorsiflexion, a phase of powered plantarflexion follows for push-off. From push-off to the next heel strike is the swing phase. Dorsiflexion during swing phase positions the toe by raising the toe to prevent stubbing. “Powered” as opposed to “controlled” in the context of dorsiflexion and plantarflexion refers to net work being performed by the energy-harvesting system to actively actuate the joint and foot, such as to dorsiflex or plantarflex the foot. “Controlled” can refer to net work being done on the energy-harvesting system (negative work) and rotation that encounters an amount of designed resistance. The resistance can be provided by the restrictors in the flow paths to the receivers, for example.

The high pressure accumulators 304 and 306 are capable of storing energy to perform work during the powered plantarflexion and swing phases as further described. The stored energy comes because of the ground reaction forces generated by the weight of the body being applied on the foot, which transfers the force to each piston/chamber unit sequentially through the gait cycle. The weight places the pistons under a high load which is sufficient to overcome the pressure in the high pressure accumulators 304, 308. The reservoirs 302 and 306 coupled with the restrictors can provide resistance to rotation thus providing a controlled rotation about the ankle during controlled plantarflexion and controlled dorsiflexion.

As can be seen in FIG. 5, in the controlled plantarflexion phase and starting about heel strike, both high pressure posterior and anterior accumulators 304 and 308 are essentially empty and both posterior and anterior reservoirs 302 and 306 are full. Fluid displacement is generally not occurring about the time of heel strike. After heel strike, and still during controlled plantarflexion, it can be seen that fluid is transferred into the posterior high pressure accumulator 304 from the posterior chamber 332, and fluid is transferred out of the anterior reservoir 306 to the anterior chamber 334. Thus, resulting in net negative work on the joint and energy being stored. During controlled plantarflexion, a human ankle will behave as a linear spring. The energy-harvesting ankle implements a mechanical impedance control scheme wherein ankle angle throughout plantarflexion increases torque on the joint. Selecting the posterior valves 322 and 302 to closed causes the posterior piston 336 to charge the posterior high-pressure accumulator 304 during controlled plantarflexion. Valve 326 is open to allow the anterior piston 338 volume to compensate via the low pressure reservoir 306. A spring-type accumulator can be used for the posterior high pressure accumulator 304 to serve linear-impedance behavior while the valves are in controlled plantarflexion.

About midstance, controlled plantarflexion ends and controlled dorsiflexion begins during “rollover.” During controlled dorsiflexion, it can be seen that fluid is transferred into the anterior high pressure accumulator 308 from the anterior chamber 334, and fluid is transferred out of the posterior reservoir 302 into the posterior chamber 332. Thus, resulting in net negative work on the joint and energy being stored. During controlled dorsiflexion, the human ankle functions as a nonlinear spring which stores energy in tendon structures to support powered plantarflexion later. The energy-harvesting ankle control scheme will select valves 326 and 328 closed to communicate the anterior piston 338 with the anterior high pressure accumulator 308. Valve 322 is open to allow the posterior piston 336 volume to compensate via the low pressure reservoir 302. The high pressure accumulator 308 can be designed as a spring or gas-charged accumulator with a nonlinear response. Therefore, the function of the human ankle during rollover (increasing stiffness and energy storage) is emulated by the energy-harvesting ankle by enabling the correct valve state.

After a period of controlled dorsiflexion, the heel begins to lift off the ground and powered plantarflexion begins about such time. In powered plantarflexion, the energy-harvesting system is performing work to plantarflex the foot to provide power to propel the body forward. During powered plantarflexion, it can be seen that the high pressure anterior accumulator 308 is transferring fluid to the anterior chamber 334, thus, causing plantarflexion and the transfer of fluid from the posterior chamber 332 to the posterior reservoir 302. Thus, resulting in net positive work performed by the joint. During powered plantarflexion, the human ankle behaves as a torque source, providing power (through muscle contraction and stored tendon energy) to the ankle and accelerating the body and leg upward and forward into swing phase. Therefore, the energy-harvesting ankle will behave as a torque source by pulse-width-modulating the solenoid-driven valve 328 of the anterior high pressure accumulator 308 into the anterior actuator piston 338. Thus, providing the correct amount of power at the desired rate through a torque controller with feedforward friction and inertia terms.

About push-off, powered plantarflexion ends, and swing with dorsiflexion begins. During swing, the high pressure posterior accumulator 304 is transferring fluid to the posterior chamber 332, thus, causing dorsiflexion and the transfer of fluid from the anterior chamber 334 to the low pressure anterior receiver 306. Thus, resulting in net positive work performed by the joint. During swing phase, the human ankle is repositioned to prepare for heel-strike. The energy-harvesting ankle engages a position-controller to emulate this behavior by dorsiflexing the foot. Dorsiflexion of the foot provides toe clearance. Valves 302 and 328 are modulated by the controller to drive the ankle back to the neutral position.

After swing phase, both the posterior and anterior high pressure accumulators 304, 308 are essentially emptied and have released their energy, and both the posterior and anterior reservoirs 302, 306 are essentially full in preparation for the next cycle. The determination of the different phases to correctly time the automatic opening and closing of the shut-off valves is carried out via the microprocessor 370 based on inputs received from the sensing device 301. As the control system is largely one of selecting appropriate valve timing relative to a gait cycle (low-level control is dominantly mechanical); at a higher level, a finite state machine accurately determines each phase of gait and appropriate control state. In one embodiment, the rate of change of the ankle torque is selected as the variable to define the timing of the pulse.

The sensing device can be programmed to use strain gauges to determine torque, rate of change of torque, and the axial force (load-determining sensor). A magnetic hall-effect encoder can be used to determine joint angle (displacement-determining sensor), and the rate of change of joint angle. The different phases of gait can be defined. Torque refers to the torque experienced at the pivot location 330. Joint angle can be described as the angle created between a line parallel to the pylon 309 and a line parallel to the longitudinal axis of the foot 315. Axial force is the vertical component of force passing through the pivot.

The logic instructions for determining the state can be implemented in a variety of hardware, software, and combined hardware/software configurations. In some embodiments, the control logic is implemented by the microprocessor 370 and memory 364. The memory can include a random access memory (“RAM”) and an electronically erasable, programmable, read-only memory (“EEPROM”) or other non-volatile memory (e.g., flash memory) or persistent storage. The RAM may be a volatile form of memory for storing program instructions that are accessible by the microprocessor. The microprocessor is configured to operate in accordance with logic instructions. Hardware or software may implement logic instructions to 1) determine when the different phases of gait exist, 2) determine the transition between the different phases of gait, and 3) open or close certain valves depending on the phase of gait or when a transition is deemed to occur.

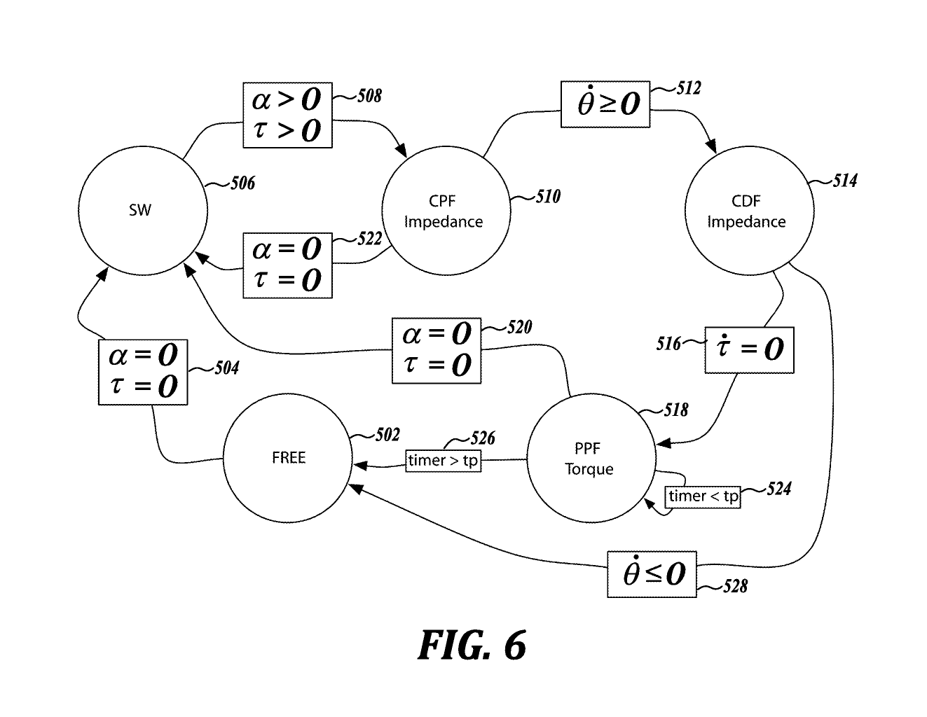

Referring to FIG. 6, a finite-state diagram is illustrated for defining the phases of gait and representative logic instructions for transitioning from state to state. The logic instructions for transitioning from state to state can be implemented in the form of hardware or software. Table 1 summarizes the energy-harvesting ankle sensory inputs. One or more load-determining sensors are used to measure the axial force and torque, while a displacement-determining sensor is used to measure the angle. Angular rate and torque rate are derived values. Time derivatives of variables are computed discreetly by the microprocessor. The energy-harvesting ankle can measure real-time dynamic load measurements in the prosthesis as inputs to the control algorithm described in FIG. 6. The transducer described in U.S. Pat. No. 7,886,618, incorporated herein expressly by reference in its entirety, can be adapted to be used in the energy-harvesting ankle. A suitable sensor device may be in a form of a pyramid adapter that incorporates silicon strain gauges to monitor moments (such as ±150 N-m in sagittal and coronal planes) and axial forces (such as ±510 N) in the prosthesis.

TABLE 1

| Parameter | Variable | Sensor |
|---|---|---|
| Joint Angle, Angular rate | θ | Magnetic hall-effect encoder |
| | δθ/δt | |
| Joint Torque, Torque rate | τ | Strain gauge sensors |
| | δτ/δt | |
| Axial Force | α | Strain gauge sensors |

Starting at the swing state 506, to enter the controlled plantarflexion state 510 from the swing state 506 requires that the axial force α is greater than 0 and the joint torque τ is greater than 0, block 508. To enter the swing state 506 from the controlled plantarflexion state 510 requires the axial force α equals 0 and the joint torque τ equals 0, block 522. To enter the controlled dorsiflexion state 514 from the controlled plantarflexion state 510 requires a rate of change in joint angle greater than or equal to 0, block 512. To enter the powered plantarflexion state 518 from the controlled dorsiflexion state 514 requires the rate of change in joint torque equal to 0, block 516. To enter the free state 502 from the controlled dorsiflexion state 514 requires the rate of change in joint angle be less than or equal to 0, block 528. Free state refers to a state where both chambers are open to the respective reservoir. To enter the swing state 506 from the powered plantarflexion state 518 requires the axial force be equal to 0 and the joint torque be equal to 0, block 520. To enter the free state 502 from the powered plantarflexion state 518 requires the time in the powered plantarflexion state to be greater than a setpoint, block 526. That is, the powered plantarflexion state 518 can time out when a timer is less than or equal to the setpoint, block 524. To enter the swing state 506 from the free state 502 requires the axial force be equal to 0 and the joint torque equal to 0. The shut-off valves 322, 302, 326, and 328 of FIG. 4 can be programmed according to open or close automatically according to the state diagram. In one embodiment, the valve states (open or closed) of the energy-harvesting system of FIG. 4 are shown in Table 2. When in the open position, the valves may be pulsed (cycled between open and closed).

TABLE 2

| Valve | CPF | CDF | PPF | SW |
|---|---|---|---|---|
| V1 (322) | Closed | Open | Open | Closed |
| V2 (302) | Closed | Closed | Closed | Open |
| V3 (326) | Open | Closed | Closed | Open |
| V4 (328) | Closed | Closed | Open | Closed |

The state diagram of FIG. 6 is not limiting. It should be understood that FIG. 6 applies to a particular joint, namely an ankle, with a particular energy-harvesting system including two antagonistic chamber/piston units. Furthermore, it should be understood that not all states may be programmed into the joint device, and fewer or more states may also be programmed. For example, a joint device can have one or more states selected from a swing state (positioning), a controlled plantarflexion state, a controlled dorsiflexion state, a powered plantarflexion state, and a free state or any combination thereof. A swing state for positioning the foot can be viewed as powered dorsiflexion. It should be understood that plantarflexion and dorsiflexion generally refer to the movement of the foot with respect to the ankle. However, in the context of other joints, movement is referred to as flexion and extension. Other joints may have powered flexion, powered extension, controlled flexion, controlled extension, or any combination thereof. The chamber/piston units can be configured to power any joint in either flexion or extension. In general, for the joints described herein, there are periods where the net negative work on the joint will be sufficiently negative (such as above a threshold) to trigger energy storage, periods where the net negative work on the joint will be below the negative work threshold, and periods where the joint performs net positive work. The flow controllers of an energy-harvesting system disclosed herein can be programmed to store energy when net negative work is above a certain threshold. When the net negative work is below the threshold, the hydraulic fluid is exchanged between the chamber and the reservoir.

The design criteria for the energy-harvesting systems can be determined on a case-by-case basis. For example, the high pressure and reservoir pressures, as well as damping factors, can be determined based on certain variable design criteria. As an example, the physical constraint defining the high pressure accumulator for powered plantarflexion accumulator may include an ankle acceleration in the range of 300 radians/s². In order to accelerate a 2.5 kg prosthetic system (foot, ankle, pylon, and socket) around the metatarsal area of a 27 cm foot (15 cm from ankle mass to toe) at this rate 300 (radians/s²), over an anthropometric distance of 28 degrees, requires 11 Joules of energy. Therefore, the high-pressure accumulator for powered plantarflexion can be capable of storing greater than 11 Joules. That energy should be applied over a period of approximately 80 ms; requiring a peak power of just under 140 W.

The volume needed in the accumulators can be based upon the knowledge of ankle position through stance phase, combined with piston area and distance to ankle pivot. For example, during controlled dorsiflexion, when the ankle should harvest 11 Joules, the ankle travels approximately 15 degrees. Assuming, a piston area of 3.6 cm² that acts at a distance of 2.4 cm from the pivot; therefore, the piston travels approximately 6 mm of linear displacement from a 15-degree sweep and 2.16 mL of fluid is displaced in the high pressure accumulator 308. The pressure required for the high pressure accumulator 308 to store 11 J in 2.16 mL is: 11 Joules/2.16 mL=5 MPa. Storage of 25 Joules is estimating over a 50% application energy loss and results in an 11.5 MPa pressure. The remaining hydraulic features can be designed similarly, with the posterior hydraulic system driving dorsiflexion in swing phase. Further design criteria for an ankle joint may include a range of motion of about 10° of dorsiflexion to 20° of plantarflexion. The torque can be about 1.6 N*m/kg of body mass. The angular velocity can be about 1.5 rad/s. The power and energy can be about 11 joules with peak power of 140 W.

While a description of an energy-harvesting system has been shown to be incorporated into an ankle joint with respect to FIG. 4, other energy-harvesting system depicted in FIGS. 1-3 can also be incorporated into an ankle joint or other joints. Also, the energy-harvesting system described for an ankle joint can also be incorporated into a hip joint, shoulder, elbow, wrist, or a legged nonhuman robot. In other embodiments, an energy-harvesting system for an ankle joint does not require the use of two opposed chambers.

The system of FIG. 1 showing a single chamber 102 can be incorporated into an ankle joint to provide powered plantarflexion, powered dorsiflexion, or both. The single chamber 102 may be placed anteriorly, posteriorly, or in the center of the joint. The valves 108, 110 are appropriately controlled to store energy in the high pressure accumulator 114 during periods of net negative work above a threshold, and release the energy to perform positive work. Likewise, the two chamber/piston unit, two accumulator energy-harvesting systems of FIGS. 2 and 3 may also be incorporated into an ankle joint. The valves in those systems are appropriately controlled to store energy in the high pressure accumulator during periods of net negative work above a threshold, and release the energy during periods when it is desired for the ankle to perform positive work. Further, the energy-harvesting system can be distributed across two or more joints. For example, the energy stored from the ground forces on the foot can be used to power other joints besides the ankle, including the knee, hip, shoulder, elbow, or wrist.

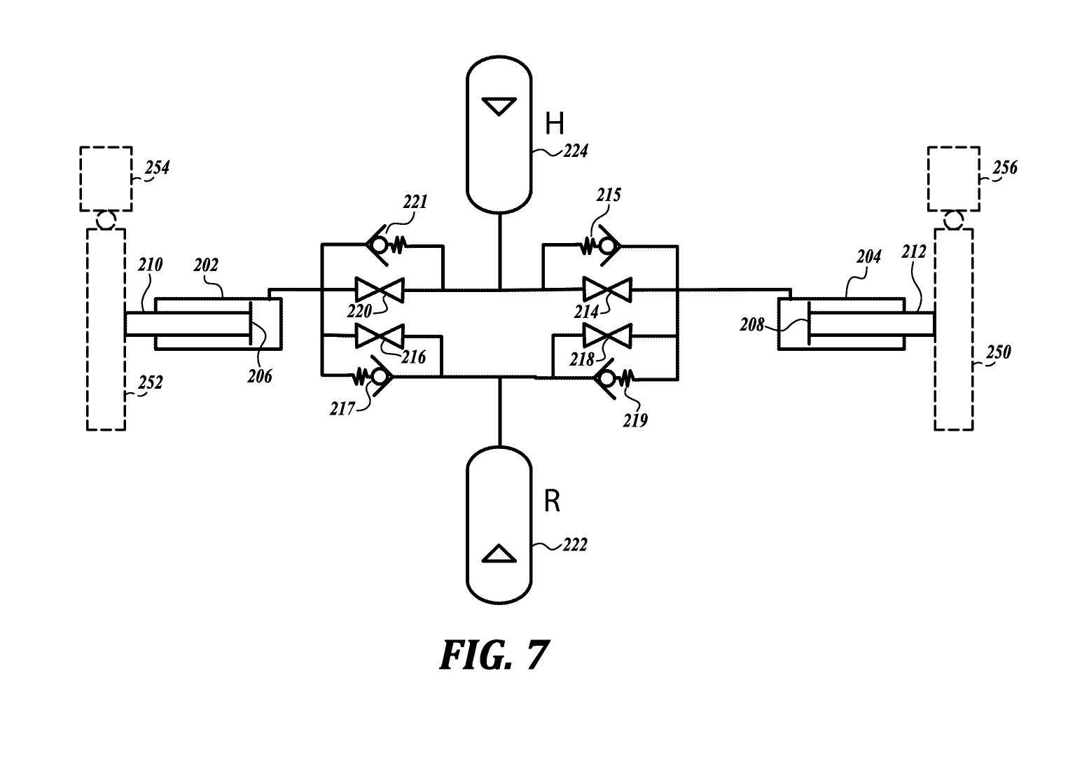

FIG. 7 shows a schematic illustration of an energy-harvesting system distributed across two joints. The energy-harvesting system is similar to the system of FIG. 3 with the following modifications. A first chamber/piston unit 202 is placed at a first joint and a second chamber/piston unit 204 is placed at a second joint. The first joint has a limb 252 (or member) that can flex or extend with respect to another limb 254 (or member). The second joint has a limb 250 (or member) that can flex or extend with respect to another limb 256 (or member). The first chamber/piston unit 202 in contact with the first limb 252 is the recipient of the net negative work above a threshold. That is, the first chamber/piston unit 202 is used to harvest energy in the accumulator 224, and the second chamber/piston unit 204 releases the energy from the accumulator to power the different limb 250.

Based on the foregoing, methods and joints are disclosed for harvesting energy and reapplying the energy. The following are representative and not meant to be limiting.

Methods of harvesting and selectively reapplying energy to a prosthetic joint are disclosed. The methods may include providing a prosthetic joint. The prosthetic joint may include at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the chamber, accumulator and reservoir; a load-determining sensor; a displacement-determining sensor; a microprocessor configured to actuate one or more flow controllers based upon a load-determining sensor input, a displacement-determining sensor input, a product of the load-determining sensor input and the displacement-determining sensor input, any time derivative thereof, or any combination thereof. The method further includes displacing fluid from the chamber to the accumulator during periods of a threshold negative work on the joint; and displacing fluid from the accumulator to the chamber to allow the joint to perform positive work.

In some embodiments, the method may further include displacing fluid from the chamber to the reservoir during periods below the threshold negative work.

In some embodiments, the prosthetic joint is an ankle joint, and the ankle joint is connected to a prosthetic foot and a pylon, wherein the ankle joint allows rotation of the prosthetic foot with respect to the pylon.

In some embodiments, the method may further include determining a flow controller state by determining a swing positioning state, a controlled plantarflexion state, a controlled dorsiflexion state, and a powered plantarflexion state.

In some embodiments, the method may further include storing energy in the accumulator during controlled dorsiflexion, or during controlled plantarflexion, or during both, and returning energy during powered plantarflexion.

In some embodiments, the method may further include returning energy during swing positioning.

In some embodiments, swing positioning includes dorsiflexing the foot and elevating the toe.

In some embodiments, the method may further include determining conditions to transition from the swing positioning state to the controlled plantarflexion state, conditions to transition from the controlled plantarflexion state to the controlled dorsiflexion state, conditions to transition from the controlled dorsiflexion state to the powered plantarflexion state, and conditions to transition from the powered plantarflexion state to the swing positioning state.

In some embodiments, in the swing positioning state, fluid is displaced from a posterior accumulator to a posterior chamber, and fluid is displaced from an anterior chamber to an anterior reservoir; in the controlled plantarflexion state, fluid is displaced from the posterior chamber to the posterior accumulator, and fluid is displaced from the anterior reservoir to the anterior chamber; in the controlled dorsiflexion state, fluid is displaced from the posterior reservoir to the posterior chamber, and fluid is displaced from the anterior chamber to the anterior accumulator; and in the powered plantarflexion state, fluid is displaced from the posterior chamber to the posterior reservoir, and fluid is displaced from the anterior accumulator to the anterior chamber.

In some embodiments, the threshold negative work is performed when a limb connected to the joint is applied on a ground surface to generate a ground reaction force greater than a pressure in the accumulator.

In some embodiments, the flow controllers include one or more automatically operated shut-off valves.

In some embodiments, the method further includes passing fluid through a restrictor when displacing fluid from the chamber to the reservoir.

In some embodiments, the method further includes producing the negative work above the threshold by contacting a limb connected to the joint on a surface to generate a ground reaction force.

In some embodiments, the displacement-determining sensor is an angle-determining sensor.

In some embodiments, the joint is a prosthetic knee joint.

In some embodiments, the method further includes storing energy in the accumulator when sitting from a standing position and returning energy when standing from a sitting position.

In some embodiments, the method further includes storing energy in the accumulator during descending and returning energy during ascending.

In some embodiments, the flow controllers are pulsed open during displacing fluid from the accumulator to the chamber.

Methods of harvesting energy from a first joint and selectively reapplying the energy to a second joint are disclosed. The methods include providing an energy-harvesting hydraulic system including at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the system; a load-determining sensor; a displacement-determining sensor; a microprocessor configured to actuate one or more flow controllers based upon a load-determining sensor input, a displacement-determining sensor input, a product of the load-determining sensor input and the displacement-determining sensor input, any time derivative thereof, or any combination thereof. The methods include displacing fluid from the chamber to the accumulator during periods of a threshold negative work on a first joint; and displacing fluid from the accumulator to the chamber to allow a second joint to perform positive work.

In some embodiments, the first joint is an ankle and the second joint is a knee, or the first joint is the knee and the second joint is the ankle.

Prosthetic joints are disclosed. The joints may include a hydraulic system, including at least one chamber; at least one accumulator configured to store hydraulic fluid at a high pressure; at least one reservoir configured to store hydraulic fluid at a low pressure; one or more fluid flow paths connecting the chamber to the accumulator and the reservoir, flow controllers in the fluid flow paths, and fluid distributed throughout the hydraulic system; a load-determining sensor; a displacement-determining sensor; and a microprocessor configured to actuate one or more flow controllers based upon a load-determining sensor input, a displacement-determining sensor input, a product of the load-determining sensor input and the displacement-determining sensor input, any time derivative thereof, or any combination thereof, wherein one or more flow controllers are configured to control displacing fluid from the chamber to the accumulator during periods of a threshold negative work, and one or more flow controllers are configured to control displacing fluid from the accumulator to the chamber to perform positive work.

In some embodiments, the joint may further include a piston in the chamber, wherein a limb is actuated by the piston during displacing fluid from the accumulator to the chamber.

In some embodiments, the limb actuates the piston during displacing fluid from the chamber to the accumulator.

In some embodiments, the joint may further include a cam and cam follower, wherein the cam follower is in contact with the cam, and the cam follower is connected to the piston.

In some embodiments, the cam includes an involute cam surface.

In some embodiments, the joint further includes a pivot, wherein the pivot rotates a first prosthetic limb with respect to a second prosthetic limb.

In some embodiments, the first prosthetic limb is a prosthetic foot, and the second prosthetic limb includes a pylon and socket.

In some embodiments, the joint further includes a first and second accumulator, a first and second reservoir, and a first and second chamber, wherein the first and second chambers are placed on opposite sides of a pivot, and the first chamber includes flow paths to the first accumulator and the first reservoir, and the second chamber includes flow paths to the second accumulator and the second reservoir.

In some embodiments, a fluid flow path from each chamber to the accumulator includes, in parallel, an automatically operated shut-off valve and a check valve, wherein the check valve is configured to allow flow from the chamber to the accumulator and obstruct flow from the accumulator to the chamber.

In some embodiments, a fluid flow path from each chamber to the reservoir includes an automatically operated shut-off valve and, in parallel, a restrictor and a check valve, wherein the check valve is configured to allow flow from the reservoir to the chamber and obstruct flow from the chamber to the reservoir.

In some embodiments, the load-determining sensor is a strain gauge.

In some embodiments, the load-determining sensor is a pressure transducer.

In some embodiments, the displacement-determining sensor is a potentiometer.

In some embodiments, the displacement-determining sensor is a hall effect sensor.

In some embodiments, the flow controllers include a solenoid valve.

Prosthetic joints are disclosed that may include a first and second connector and a pivot device that allows the first and second connector to rotate with respect to each other, wherein the first connector is configured to attach to a first prosthetic member and the second connector is configured to attach to a second prosthetic member; a first and second chamber, wherein the chambers are disposed on opposite sides of the pivot device; a first and second piston positioned in the first and second chamber, wherein the pistons are positioned to actuate the rotation of the joint; an accumulator configured to store hydraulic fluid at a high pressure, wherein the accumulator connects to each chamber through a flow path including, in parallel, a shut-off valve and a check valve, wherein the check valve is configured to allow flow from each respective chamber to the accumulator and obstruct flow from the accumulator to each respective chamber; a reservoir configured to store hydraulic fluid at a low pressure, wherein the reservoir connects to each chamber through a flow path including a shut-off valve; a load-determining sensor; a displacement-determining sensor; a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from one chamber at a time to the accumulator during periods of a threshold negative work on the joint, and displacing fluid from the accumulator to one chamber at a time to allow the joint to perform positive work.

Prosthetic joints are disclosed that may include a first and second connector and a pivot device that allows the first and second connector to rotate with respect to each other, wherein the first connector is configured to attach to a first prosthetic member and the second connector is configured to attach to a second prosthetic member; a first and second chamber, wherein the chambers are disposed on opposite sides of the pivot device; a first and second piston positioned in the first and second chamber, wherein the pistons are positioned to actuate the rotation of the joint; a first and second accumulator configured to store hydraulic fluid at a high pressure, wherein the first accumulator connects to the first chamber through a flow path including, in parallel, a shut-off valve and a check valve, wherein the check valve is configured to allow flow from the first chamber to the first accumulator and obstruct flow from the first accumulator to the first chamber, and the second accumulator connects to the second chamber through a flow path including, in parallel, a shut-off valve and a check valve, wherein the check valve is configured to allow flow from the second chamber to the second accumulator and obstruct flow from the second accumulator to the second chamber; a first and second reservoir configured to store hydraulic fluid at a low pressure, wherein the first reservoir connects to the first chamber through a flow path including, in parallel, shut-off valve and a check valve configured to allow flow from the first reservoir to the first chamber and obstruct flow from the first chamber to the first reservoir, and the second reservoir connects to the second chamber through a flow path including a shut-off valve and a check valve configured to allow flow from the second reservoir to the second chamber and obstruct flow from the second chamber to the second reservoir; a load-determining sensor; a displacement-determining sensor; a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from each chamber to the respective accumulator during periods of a threshold negative work on the joint, and for displacing fluid from each accumulator to the respective chamber to allow the joint to perform positive work.

Prosthetic joints are disclosed that may include a hydraulic system including: at least one chamber; at least one accumulator configured to store hydraulic fluid at a high pressure; at least one reservoir configured to store hydraulic fluid at a low pressure; one or more fluid flow paths connecting the chamber to the accumulator and reservoir, and flow controllers in the fluid flow paths; and hydraulic fluid in the system. The joints may further include a load-determining sensor; a displacement-determining sensor; a microprocessor to actuate the flow controllers based upon a load-determining sensor input, a displacement-determining sensor, any product of the load-determining sensor input and the displacement-determining sensor input, any time derivative thereof, or any combination thereof, wherein the flow controllers are configured to displace fluid from the chamber to the accumulator during periods of a threshold negative work, and the flow controllers are configured to displace fluid from the accumulator to the chamber to perform positive work, and wherein the threshold negative work is performed on a first joint and the positive work is performed by a second joint different from the first joint.

Some embodiments of the prosthetic joints include flow controllers that are further configured to displace fluid from the chamber to the reservoir during periods below the threshold negative work.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of harvesting and selectively reapplying energy to a prosthetic joint, comprising:
providing a prosthetic joint, comprising:
at least one chamber;
at least one accumulator configured to store hydraulic fluid at a high pressure;
at least one reservoir configured to store hydraulic fluid at a low pressure;
one or more fluid flow paths connecting the chamber to the accumulator and the reservoir, flow controllers in the fluid flow paths, and fluid distributed throughout the chamber, accumulator and reservoir;
a load-determining sensor;
a displacement-determining sensor;
a microprocessor configured to actuate one or more flow controllers based upon a load-determining sensor input, a displacement-determining sensor input, a product of the load-determining sensor input and the displacement-determining sensor input, any time derivative thereof, or any combination thereof;
displacing fluid from the chamber to the accumulator during periods of a threshold negative work on the joint; and
displacing fluid from the accumulator to the chamber to allow the joint to perform positive work.

2. The method of claim 1, further comprising displacing fluid from the chamber to the reservoir during periods below the threshold negative work.

3. The method of anyone of claims 1-2, wherein the prosthetic joint is an ankle joint, and the ankle joint is connected to a prosthetic foot and a pylon, wherein the ankle joint allows rotation of the prosthetic foot with respect to the pylon.

4. The method of anyone of claims 1-3, further comprising determining a flow controller state by determining a swing positioning state, a controlled plantarflexion state, a controlled dorsiflexion state, and a powered plantarflexion state.

5. The method of claim 4, further comprising storing energy in the accumulator during controlled dorsiflexion, or during controlled plantarflexion, or during both, and returning energy during powered plantarflexion.

6. The method of claim 5, further comprising returning energy during swing positioning.

7. The method of claim 6, wherein positioning includes dorsiflexing the foot and elevating the toe.

8. The method of claim 4, further comprising determining conditions to transition from the swing positioning state to the controlled plantarflexion state, conditions to transition from the controlled plantarflexion state to the controlled dorsiflexion state, conditions to transition from the controlled dorsiflexion state to the powered plantarflexion state, and conditions to transition from the powered plantarflexion state to the swing positioning state.

9. The method of claim 4, wherein,
in the swing positioning state, fluid is displaced from a posterior accumulator to a posterior chamber, and fluid is displaced from an anterior chamber to an anterior reservoir;
in the controlled plantarflexion state, fluid is displaced from the posterior chamber to the posterior accumulator, and fluid is displaced from the anterior reservoir to the anterior chamber;
in the controlled dorsiflexion state, fluid is displaced from the posterior reservoir to the posterior chamber, and fluid is displaced from the anterior chamber to the anterior accumulator;
in the powered plantarflexion state, fluid is displaced from the posterior chamber to the posterior reservoir, and fluid is displaced from the anterior accumulator to the anterior chamber.

10. The method of anyone of claims 1-9, wherein the threshold negative work is performed when a limb connected to the joint is applied on a ground surface to generate a ground reaction force greater than a pressure in the accumulator.

11. The method of anyone of claims 1-11, wherein the flow controllers include one or more automatically operated shut-off valves.

12. The method of claim 2, further comprising passing fluid through a restrictor when displacing fluid from the chamber to the reservoir.

13. The method of anyone of claims 1-12, further comprising producing the negative work above the threshold by contacting a limb connected to the joint on a surface to generate a ground reaction force.

14. The method of anyone of claims 1-13, wherein the displacement-determining sensor is an angle-determining sensor.

15. The method of anyone of claims 1-14; wherein the joint is a prosthetic knee joint.

16. The method of claim 15; further comprising storing energy in the accumulator when sitting from a standing position and returning energy when standing from a sitting position.

17. The method of claim 15; further comprising storing energy in the accumulator during descending and returning energy during ascending.

18. The method of anyone of claims 1-15, wherein the flow controllers are pulsed open during displacing fluid from the accumulator to the chamber.

19. A method of harvesting energy from a first joint and selectively reapplying the energy to a second joint, comprising:
providing an energy-harvesting hydraulic system, comprising:
at least one chamber;
at least one accumulator configured to store hydraulic fluid at a high pressure;
at least one reservoir configured to store hydraulic fluid at a low pressure;
one or more fluid flow paths connecting the chamber to the accumulator and the reservoir, flow controllers in the fluid flow paths, and fluid distributed throughout the system;
a load-determining sensor;
a displacement-determining sensor;
a microprocessor configured to actuate one or more flow controllers based upon a load-determining sensor input, a displacement-determining sensor input, a product of the load-determining sensor input and the displacement-determining sensor input, any time derivative thereof, or any combination thereof; and
displacing fluid from the chamber to the accumulator during periods of a threshold negative work on a first joint; and
displacing fluid from the accumulator to the chamber to allow a second joint to perform positive work.

20. The method of claim 19, wherein the first joint is an ankle and the second joint is a knee, or the first joint is the knee and the second joint is the ankle.

21. A prosthetic joint, comprising
a hydraulic system, comprising:
at least one chamber;
at least one accumulator configured to store hydraulic fluid at a high pressure;
at least one reservoir configured to store hydraulic fluid at a low pressure;
one or more fluid flow paths connecting the chamber to the accumulator and the reservoir, flow controllers in the fluid flow paths, and fluid distributed throughout the hydraulic system;
a load-determining sensor;
a displacement-determining sensor; and
a microprocessor configured to actuate one or more flow controllers based upon a load-determining sensor input, a displacement-determining sensor input, a product of the load-determining sensor input and the displacement-determining sensor input, any time derivative thereof, or any combination thereof, wherein one or more flow controllers are configured to control displacing fluid from the chamber to the accumulator during periods of a threshold negative work, and one or more flow controllers are configured to control displacing fluid from the accumulator to the chamber to perform positive work.

22. The joint of claim 21, further comprising a piston in the chamber, wherein a limb is actuated by the piston during displacing fluid from the accumulator to the chamber.

23. The joint of anyone of claims 21-22, wherein the limb actuates the piston during displacing fluid from the chamber to the accumulator.

24. The joint of claim 22, further comprising a cam and cam follower, wherein the cam follower is in contact with the cam, and the cam follower is connected to the piston.

25. The joint of claim 24, wherein the cam includes an involute cam surface.

26. The joint of anyone of claims 21-25, further comprising a pivot, wherein the pivot rotates a first prosthetic limb with respect to a second prosthetic limb.

27. The joint of claim 26, wherein the first prosthetic limb is a prosthetic foot, and the second prosthetic limb includes a pylon and socket.

28. The joint of anyone of claims 21-27, further comprising a first and second accumulator, a first and second reservoir, and a first and second chamber, wherein the first and second chambers are placed on opposite sides of a pivot, and the first chamber includes flow paths to the first accumulator and the first reservoir, and the second chamber includes flow paths to the second accumulator and the second reservoir.

29. The joint of anyone of claims 21-28, wherein a fluid flow path from each chamber to the accumulator includes, in parallel, an automatically operated shut-off valve and a check valve, wherein the check valve is configured to allow flow from the chamber to the accumulator and obstruct flow from the accumulator to the chamber.

30. The joint of claim 28, wherein a fluid flow path from each chamber to the reservoir includes an automatically operated shut-off valve and, in parallel, a restrictor and a check valve, wherein the check valve is configured to allow flow from the reservoir to the chamber and obstruct flow from the chamber to the reservoir.

31. The joint of anyone of claims 21-30, wherein the load-determining sensor is a strain gauge.

32. The joint of anyone of claims 21-30, wherein the load-determining sensor is a pressure transducer.

33. The joint of anyone of claims 21-32, wherein the displacement-determining sensor is a potentiometer.

34. The joint of anyone of claims 21-32, wherein the displacement-determining sensor is a hall effect sensor.

35. The joint of anyone of claims 21-34, wherein the flow controllers include a solenoid valve.

36. A prosthetic joint, comprising:
a first and second connector and a pivot device that allows the first and second connector to rotate with respect to each other, wherein the first connector is configured to attach to a first prosthetic member and the second connector is configured to attach to a second prosthetic member;
a first and second chamber, wherein the chambers are disposed on opposite sides of the pivot device;
a first and second piston positioned in the first and second chamber, wherein the pistons are positioned to actuate the rotation of the joint;
an accumulator configured to store hydraulic fluid at a high pressure, wherein the accumulator connects to each chamber through a flow path including, in parallel, a shut-off valve and a check valve, wherein the check valve is configured to allow flow from each respective chamber to the accumulator and obstruct flow from the accumulator to each respective chamber;
a reservoir configured to store hydraulic fluid at a low pressure, wherein the reservoir connects to each chamber through a flow path including a shut-off valve;
a load-determining sensor;
a displacement-determining sensor;
a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from one chamber at a time to the accumulator during periods of a threshold negative work on the joint, and displacing fluid from the accumulator to one chamber at a time to allow the joint to perform positive work.

37. A prosthetic joint, comprising:
a first and second connector and a pivot device that allows the first and second connector to rotate with respect to each other, wherein the first connector is configured to attach to a first prosthetic member and the second connector is configured to attach to a second prosthetic member;
a first and second chamber, wherein the chambers are disposed on opposite sides of the pivot device;
a first and second piston positioned in the first and second chamber, wherein the pistons are positioned to actuate the rotation of the joint;
a first and second accumulator configured to store hydraulic fluid at a high pressure, wherein the first accumulator connects to the first chamber through a flow path including, in parallel, a shut-off valve and a check valve, wherein the check valve is configured to allow flow from the first chamber to the first accumulator and obstruct flow from the first accumulator to the first chamber, and the second accumulator connects to the second chamber through a flow path including, in parallel, a shut-off valve and a check valve, wherein the check valve is configured to allow flow from the second chamber to the second accumulator and obstruct flow from the second accumulator to the second chamber;
a first and second reservoir configured to store hydraulic fluid at a low pressure, wherein the first reservoir connects to the first chamber through a flow path including, in parallel, shut-off valve and a check valve configured to allow flow from the first reservoir to the first chamber and obstruct flow from the first chamber to the first reservoir, and the second reservoir connects to the second chamber through a flow path including a shut-off valve and a check valve configured to allow flow from the second reservoir to the second chamber and obstruct flow from the second chamber to the second reservoir;
a load-determining sensor;
a displacement-determining sensor;
a microprocessor configured to actuate the shut-off valves based upon a load-determining sensor input, a displacement-determining sensor input, a product of the load-determining sensor input and the displacement-determining sensor input, any time derivative thereof, or any combination thereof, for displacing fluid from each chamber to the respective accumulator during periods of a threshold negative work on the joint, and for displacing fluid from each accumulator to the respective chamber to allow the joint to perform positive work.

38. A prosthetic joint, comprising:
a hydraulic system comprising:
at least one chamber;
at least one accumulator configured to store hydraulic fluid at a high pressure;
at least one reservoir configured to store hydraulic fluid at a low pressure;
one or more fluid flow paths connecting the chamber to the accumulator and reservoir, and flow controllers in the fluid flow paths; and
hydraulic fluid in the system;
a load-determining sensor;
a displacement-determining sensor;
a microprocessor to actuate the flow controllers based upon a load-determining sensor input, a displacement-determining sensor, any product of the load-determining sensor input and the displacement-determining sensor input, any time derivative thereof, or any combination thereof, wherein the flow controllers are configured to displace fluid from the chamber to the accumulator during periods of a threshold negative work, and the flow controllers are configured to displace fluid from the accumulator to the chamber to perform positive work, and wherein the threshold negative work is performed on a first joint and the positive work is performed by a second joint different from the first joint.

39. The prosthetic joint of any one of claims 36-38, wherein the flow controllers are further configured to displace fluid from the chamber to the reservoir during periods below the threshold negative work.
